(19) United States
(12) Patent Application Publication
(10) Pub. No.: US 2009/0230024 A1
(43) Pub. Date: Sep. 17, 2009

(54) Co-production of hydrocarbons and dimethyl ether

(76) Inventors: Andre Peter Steynberg, Vanderbijlpark (ZA); Pierre Greeff, Johannesburg (ZA)

Correspondence Address: LADAS & PARRY LLP, 26 WEST 61ST STREET, NEW YORK, NY, 10023, US

(21) Appl. No.: 10/588,475

(22) PCT Filed: Feb. 3, 2005
(86) PCT No.: PCT/IB05/50449

§ 371 Date: Feb. 19, 2008

(30) Foreign Application Priority Data
Feb. 5, 2004 (US) 10542088

(51) Int. Cl. C10G 35/04; C07C 27/06

(52) U.S. Cl. 208/141; 518/705

(57) ABSTRACT

A process for co-producing hydrocarbons and dimethyl ether (DME) includes feeding a gaseous feedstock comprising hydrogen and carbon monoxide, into a threephase low temperature catalytic Fischer-Tropsch reaction stage, allowing the hydrogen and carbon monoxide partially to react catalytically in the Fischer-Tropsch reaction stage to form hydrocarbons, and obtaining a tail gas from the Fischer-Tropsch reaction stage which includes unreacted hydrogen and carbon monoxide and also carbon dioxide. The composition of at least a portion of the tail gas is adjusted to provide a DME synthesis feedstock with a syngas number (SN) between 1.8 and 2.2, where formula (I) and where [H₂], [CO] and [CO₂] respectively are the molar proportions of hydrogen, carbon monoxide and carbon dioxide in the DME synthesis feedstock. The DME synthesis feedstock is fed into a DME synthesis stage for conversion.

$$SN = \frac{[ {H}_{2} ] - [ {CO}_{2} ]}{[ CO ] + [ {CO}_{2} ]} \qquad ( I )$$

THIS INVENTION relates to co-production of hydrocarbons and dimethyl ether. In particular, the invention relates to a process for co-producing hydrocarbons and dimethyl ether (DME), and to a process for co-producing liquid fuels and light olefins.

According to one aspect of the invention, there is provided a process for co-producing hydrocarbons and dimethyl ether (DME), the process including

feeding a gaseous feedstock comprising hydrogen and carbon monoxide, into a three-phase low temperature catalytic Fischer-Tropsch reaction stage;

allowing the hydrogen and carbon monoxide partially to react catalytically in the Fischer-Tropsch reaction stage to form hydrocarbons;

obtaining a tail gas from the Fischer-Tropsch reaction stage which includes unreacted hydrogen and carbon monoxide and also carbon dioxide;

adjusting the composition of at least a portion of the tail gas to provide a DME synthesis feedstock with a syngas number (SN) between 1.8 and 2.2, where

$$SN = \frac{[ {H}_{2} ] - [ {CO}_{2} ]}{[ CO ] + [ {CO}_{2} ]}$$

and where [H₂], [CO] and [CO₂] respectively are the molar proportions of hydrogen, carbon monoxide and carbon dioxide in the DME synthesis feedstock;

feeding the DME synthesis feedstock into a DME synthesis stage; and

converting at least a portion of the DME synthesis feedstock fed to the DME synthesis stage to DME.

Typically, the Fischer-Tropsch reaction stage includes a slurry bed of a solid particulate Fischer-Tropsch catalyst suspended in a carrier liquid, with the gaseous feedstock entering the slurry bed at a low level.

The Fischer-Tropsch catalyst used in the Fischer-Tropsch reaction stage may be a shifting catalyst, e.g. an iron catalyst, and is preferably a promoted iron catalyst. The catalyst may be promoted for activity and/or selectivity.

By “shifting catalyst” is meant a hydrocarbon synthesis catalyst which, at the operating conditions of the Fischer-Tropsch reaction stage, converts more than 2% of CO passing through the reaction stage into CO₂ by the water-gas shift reaction:

CO+H₂O⇄CO₂+H₂

When employing a slurry bed in the Fischer-Tropsch reaction stage, the hydrogen and carbon monoxide react catalytically as they pass upwardly through the slurry bed, thereby to form liquid hydrocarbon products and gaseous products, with the liquid hydrocarbon products thus constituting the carrier liquid of the slurry bed.

The process typically includes withdrawing liquid hydrocarbon products and gases and vapours from the Fischer-Tropsch reaction stage, cooling the gases and vapours to condense liquid hydrocarbons and reaction water present therein and to obtain the tail gas comprising the unreacted hydrogen and carbon monoxide. Typically, the condensed liquid hydrocarbons, reaction water and tail gas are separated in, and withdrawn from, a separator vessel, with the composition of at least a portion of the tail gas then being adjusted to provide the DME synthesis feedstock with the desired syngas number.

The tail gas from the Fischer-Tropsch reaction stage thus typically includes unreacted hydrogen, unreacted carbon monoxide and gaseous products which are formed in the Fischer-Tropsch reaction stage, including CO₂, and which have not been condensed and separated from the tail gas. This tail gas typically includes small quantities of C₅-hydrocarbons. Thus carbon dioxide will be formed in the Fischer-Tropsch reaction stage by the water-gas shift reaction.

Preferably, the syngas number is between 1.85 and 2.15, more preferably between 1.9 and 2.1, e.g. about 2.

Advantageously, it is expected that the DME synthesis feedstock may include light hydrocarbons from the Fischer-Tropsch reaction stage, without the presence of the light hydrocarbons adversely affecting the DME synthesis, thus obviating the need substantially entirely to remove these light hydrocarbons from the tail gas obtained from the Fischer-Tropsch reaction stage.

Adjusting the composition of at least a portion of the tail gas from the Fischer-Tropsch reaction stage may include removing some CO₂ from said portion of the tail gas. Thus, the syngas number may be adjusted upwardly. As will be appreciated, the gaseous feedstock can be derived from a methane-containing gas such as natural gas, or it can be derived from solid carbonaceous material such as coal. When the gaseous feedstock is derived from a carbonaceous material, such as coal, it is expected that, in a preferred embodiment of the process, CO₂ will be removed from said portion of the tail gas. In an optional embodiment of the invention CO₂ is removed from said portion of the tail gas when the gaseous feedstock is derived from a methane-containing gas.

Removing some CO₂ from said portion of the tail gas may include absorbing the CO₂ in a solvent, e.g. a Benfield solution. The process may thus also include recovering the removed CO₂, by stripping the CO₂ from the solvent. This may be achieved, for example, by using a stripping gas and elevating the temperature of the solvent.

In one embodiment of the invention, the CO₂ is stripped from the solvent with a methane-containing gas and the gaseous feedstock to the Fischer-Tropsch reaction stage is derived from said methane-containing gas.

Instead, or in addition, adjusting the composition of at least a portion of the tail gas from the Fischer-Tropsch reaction stage may include adding an H₂ rich gas to said portion of the tail gas.

Adding an H₂ rich gas to said portion of the tail gas from the Fischer-Tropsch reaction stage may include reforming a portion of the gaseous feedstock to the Fischer-Tropsch reaction stage in a steam reforming stage to produce an H₂ rich reformed gas, and combining at least some of the H₂ rich reformed gas with said portion of the tail gas to provide the DME synthesis feedstock.

Typically, an installation for synthesising hydrocarbons which employs a Fischer-Tropsch synthesis stage includes a hydroprocessing facility, which in turn relies on a steam reforming facility to generate H₂ for hydroprocessing. Advantageously, the process of the invention can thus rely on such a steam reforming facility, possibly upgraded if necessary, also to provide the H₂ rich reformed gas with which the composition of said portion of the tail gas can be adjusted.

The process may include removing an H₂ containing tail gas from the DME synthesis stage, recovering an H₂ rich gas from the DME synthesis stage tail gas, and adding said H₂ rich gas to the portion of the tail gas from the Fischer-Tropsch reaction stage to provide the DME synthesis feedstock. This may be achieved by means of pressure swing adsorption (PSA) or cold separation.

Instead, adding an H₂ rich gas to said portion of the tail gas from the Fischer-Tropsch reaction stage may include subjecting a synthesis gas to the water gas shift reaction CO+H₂O⇄CO₂+H₂, and removing at least some of the CO₂ to provide the H₂ rich gas, and combining at least some of the H₂ rich gas with said portion of the tail gas to provide the DME synthesis feedstock. The CO₂ may be removed by absorption in a suitable absorbent or solvent. A suitable synthesis gas for the water gas shift reaction may be obtained from the gaseous feedstock to the Fischer-Tropsch reaction stage, or tail gas from the Fischer-Tropsch reaction stage or from the DME synthesis stage.

The process may include recycling some of the tail gas from the Fischer-Tropsch reaction stage to the Fischer-Tropsch reaction stage. The tail gas recycle may be used to increase overall Fischer-Tropsch reaction stage CO and H₂ conversion to a value of between about 30% and about 60%, preferably to a value between about 30% and about 50%. The CO and H₂ per pass conversion is in the order of about 30%. It is known that a reaction stage using promoted iron Fischer-Tropsch catalyst suffers from a rapid decline in reaction stage productivity as the per pass conversion of CO and H₂ increases. It is thus advantageous that the Fischer-Tropsch reaction stage may be operated with a low per pass conversion of CO and H₂ of between about 30% and about 50%, preferably about 30%.

The gaseous feedstock to tail gas recycle ratio will typically be about 1:1, but may vary depending on the gaseous feedstock composition.

The Fischer-Tropsch reaction stage may operate at a temperature of less than 280° C. Typically, the Fischer-Tropsch reaction stage operates at a temperature of between 160° C. and 280° C., preferably between 220° C. and 260° C., e.g. about 240° C. The Fischer-Tropsch reaction stage is thus a high chain growth, typically slurry bed, reaction stage, operating at a predetermined operating pressure in the range 10 to 50 bar, e.g. about 30 bar.

The process of the invention may include treating the hydrocarbons to provide a naphtha fraction and/or a kerosene fraction, e.g. a C₅ to a C₈ or Cg naphtha cut or fraction and a C₉ or C₁₀ to C₁₃ or C₁₄ kerosene cut or fraction. Preferably, lubricating oils and diesel are also produced. In fact, the liquid hydrocarbon product from the Fischer-Tropsch reaction stage may comprise predominantly wax. In other words, at least about 50% by mass of the liquid hydrocarbon product from the Fischer-Tropsch reaction stage may be made up of C₁₉+ hydrocarbons. This wax may be processed in a wax processing or hydroprocessing stage to give high yields of high quality lubricant base oil products and/or high value wax products. The wax processing stage may also yield a portion of the naphtha fraction, e.g. a C₅ to C₁₀ naphtha fraction.

A kerosene fraction, e.g. a C₉ to C₁₃ fraction of the condensed liquid hydrocarbons from the Fischer-Tropsch reaction stage may be treated to remove oxygenated hydrocarbons and then alkylated and subjected to a separation stage to produce linear alkyl benzene, and optionally paraffins and oxygenates. Alternatively, the kerosene fraction may serve as feedstock to a hydroformylation process to produce detergent range alcohols. The naphtha fraction may be processed to extract olefin comonomers such as 1-hexene and 1-octene, and optionally paraffins and oxygenates.

As mentioned hereinbefore, the gaseous feedstock may be derived from a methane-containing gas. Derivation of the gaseous feedstock may include reforming the methane-containing gas in a reforming stage in the presence of oxygen and steam. The reforming stage may be an autothermal reforming stage. Preferably, a low steam to carbon ratio of between about 0.2 and about 0.6, e.g. about 0.4, is used in the autothermal reforming stage. Instead, the reforming stage may be a catalytic or a non-catalytic partial oxidation stage, in which a steam to carbon ratio of 0.2 or less is typically used.

When derived from a methane-containing gas, the gaseous feedstock may comprise hydrogen and carbon monoxide in a molar ratio of between about 1.5 and about 2.3. Thus there may be an excess of H₂ above the stoichiometric requirements for hydrocarbon synthesis. When derived from a solid carbonaceous material, and relying on gasification of the solid carbonaceous material in a gasification stage, the carbonaceous feedstock typically has an H₂/CO molar ratio of between about 0.4 and about 2.1, often between 0.7 and 2.0. Thus there may be a deficit of the H₂ compared to the stoichiometric requirement for hydrocarbon synthesis.

Converting at least a portion of the DME synthesis feedstock fed to the DME synthesis stage to DME may include contacting the DME synthesis feedstock with a methanol catalyst and a methanol dehydration catalyst, thereby to produce DME and methanol. Typically, this is at elevated pressure, say between 50 bar and 100 bar, e.g. about 100 bar, usually at an operating pressure higher than the operating pressure of the Fischer-Tropsch reaction stage. The DME synthesis stage will typically require recycle of unreacted DME synthesis feedstock to obtain satisfactory conversion.

As methanol catalyst, a copper-containing catalyst is usually employed. Suitable catalysts however include compositions containing copper, zinc oxide, chromia and/or alumina and possibly other oxidic materials such as magnesia.

The produced methanol may thus be dehydrated to produce the DME. Typically, a product mixture with a DME to methanol mass ratio of approximately 2:1 is produced, which can be subjected to a rectification process to recover a DME product with the required purity. Separated methanol may be recycled to the DME synthesis stage. Thus, DME is produced in combination with methanol by a combined synthesis from the DME feedstock by use of catalysts active in the synthesis of methanol from synthesis gas and methanol dehydration. Methanol dehydration catalysts usually comprise alumina or alumina silicates as active compounds.

According to another aspect of the invention, there is provided a process for

co-producing a liquid fuel and light olefins, the process including co-producing liquid hydrocarbons and dimethyl ether (DME) from a gaseous feedstock comprising hydrogen and carbon monoxide;

treating the liquid hydrocarbons to provide a liquid fuel; and

converting at least some of the DME into light olefins.

The liquid hydrocarbons and the DME may be co-produced in accordance with the process in accordance with the first aspect of the invention. Advantageously, the DME being converted into light olefins need not be of high purity.

Treating the liquid hydrocarbons to provide a liquid fuel may include subjecting at least a portion of the liquid hydrocarbons to hydroprocessing, thereby to produce lubricants and a diesel fraction, the liquid fuel produced thus being Fischer-Tropsch derived liquid fuel and including a diesel fraction.

Treating the liquid hydrocarbons may also include producing a naphtha fraction and optionally a kerosene fraction, and converting at least some of the naphtha fraction and optionally some of the kerosene fraction with at least some of the DME into light olefins, such as ethylene and propylene. Preferably, a zeolite such as ZSM-5 or a molecular sieve catalyst, preferably a silicoalumino phosphate catalyst is used to convert the DME and a naphtha fraction and optionally a kerosene fraction into light olefins. Suitable silicoalumino phosphate catalysts include SAPO-5, SAPO-8, SAPO-11, SAPO-16, SAPO-17, SAPO-18, SAPO-20, SAPO-31, SAPO-34, SAPO-35, SAPO-36, SAPO-37, SAPO-40, SAPO-41, SAPO-42, SAPO-44, SAPO-47 and SAPO-56, the metal-containing forms thereof, and mixtures thereof.

The invention will now be described, by way of example, with reference to the two accompanying diagrammatic drawings in which

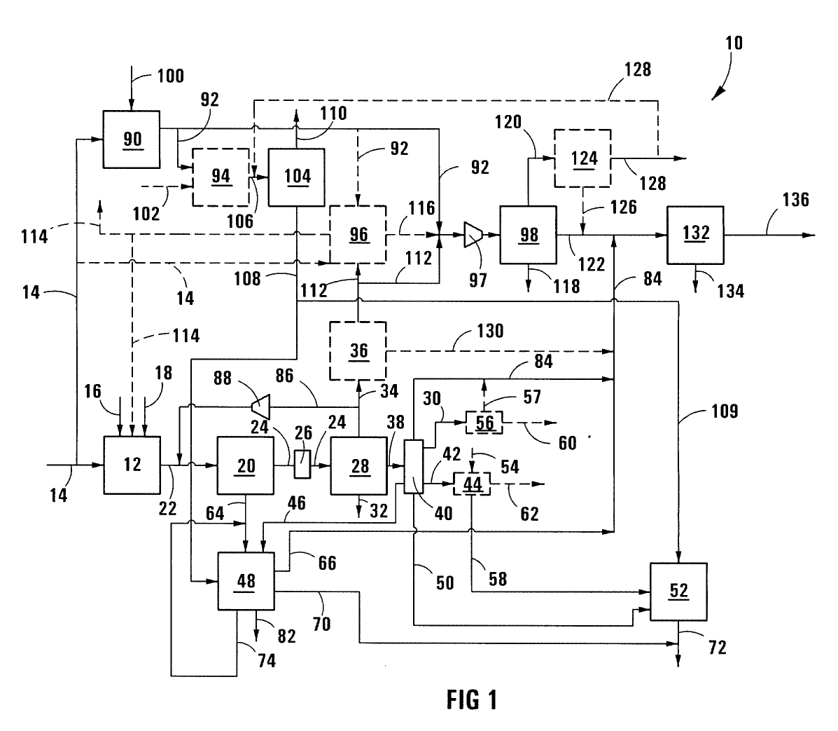

FIG. 1 shows a simplified flow sheet of a process in accordance with the invention for co-producing hydrocarbons, DME and light olefins; and

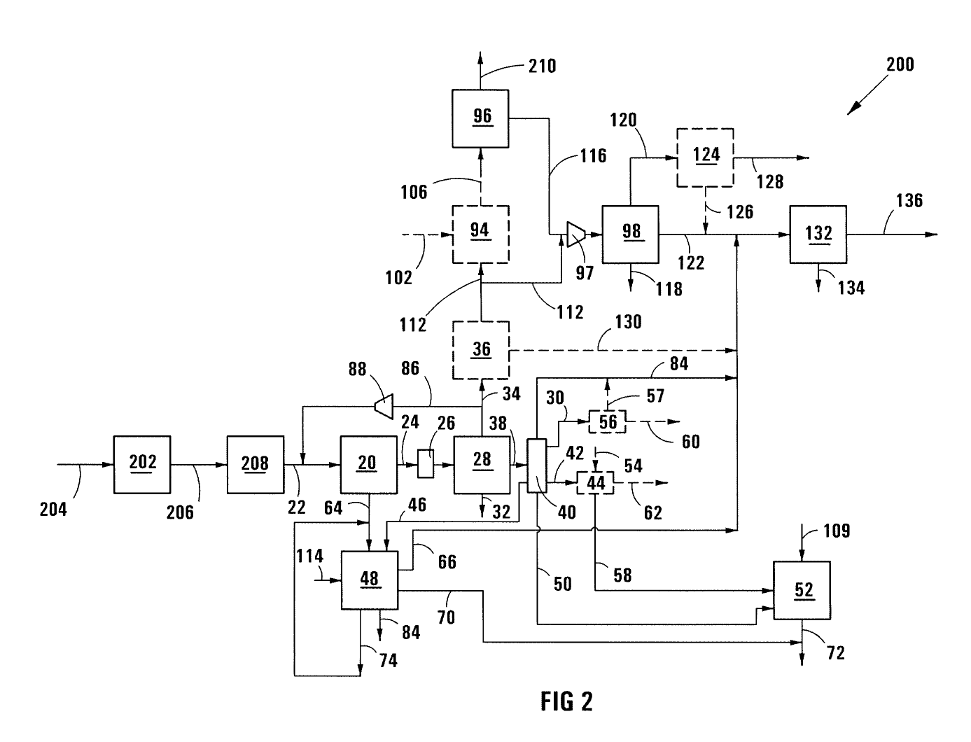

FIG. 2 shows a simplified flow sheet of another embodiment of the process in accordance with the invention for co-producing hydrocarbons, DME and light olefins.

Referring to FIG. 1 of the drawings, reference numeral 10 generally indicates a process in accordance with the invention for co-producing hydrocarbons such as Fischer-Tropsch derived lubricating oils and diesel, DME and light olefins such as ethylene and propylene.

The process 10 includes an autothermal reforming stage 12 provided with a natural gas feedstock line 14 and oxygen and steam feed lines 16, 18. The autothermal reforming stage 12 is connected to a Fischer-Tropsch reaction stage 20 by means of a syngas feed line 22. A gaseous product line 24 leads from the Fischer-Tropsch reaction stage 20 to an air cooler 26 and from the air cooler 26 to a three-phase separation stage 28. An aqueous condensate line 32 leads from the three-phase separation stage 28, a tail gas line 34 leads to an optional light hydrocarbon recovery stage 36 and a hydrocarbon condensate line 38 leads to a fractionation stage 40. From the fractionation stage 40 a naphtha fraction line 30 leads to an optional comonomer separation stage 56, a kerosene fraction line 42 leads to an alkylation stage 44, a heavies line 46 leads to a hydroprocessing stage 48, a diesel fraction line 50 leads to a diesel hydrotreating stage 52, and a light hydrocarbon line 84 is provided.

The optional comonomer stage 56 is connected to the light hydrocarbon line 84 from the fractionation stage 40 with a naphtha line 57. Comonomer product lines 60 (only one of which is shown) for comonomer product such as 1-hexene and 1-octene lead from the optional comonomer stage 56.

The optional alkylation stage 44 is provided with a benzene feed line 54 and is connected to the diesel hydrotreating stage 52 by a paraffins/oxygenates feed line 58. A linear alkyl benzene product line 62 also leads from the optional alkylation stage 44.

The Fischer-Tropsch reaction stage 20 is connected to the hydroprocessing stage 48 by means of a liquid hydrocarbon line 64. A naphtha line 66 from the hydroprocessing stage 48 joins the light hydrocarbon line 84 from the fractionation stage 40 and a diesel line 70 from the hydroprocessing stage 48 joins a diesel line 72 from the diesel hydrotreating stage 52. An unconverted heavies line 74 from the hydroprocessing stage 48 is recycled to extinction to the hydroprocessing stage 48. A lubricating oils product line 82 leads from the hydroprocessing stage 48.

A tail gas recycle line 86 and a compressor 88 are provided to recycle tail gas from the three-phase separation stage 28 to the Fischer-Tropsch reaction stage 20.

The process 10 further includes a steam reforming stage 90 also fed by the natural gas feedstock line 14. From the steam reforming stage 90, a hydrogen rich gas line 92 leads and branches to feed into an optional water-gas shift reaction stage 94, and optionally to a DME synthesis stage 98. The steam reforming stage 90 is provided with a steam feed line 100 and the optional water-gas shift reaction stage 94 is provided with a steam feed line 102.

The process 10 further includes a pressure swing adsorption stage 104 which receives gas, either from the water-gas shift reaction stage 94 or the steam reforming stage 90 if the water-gas shift reaction stage 94 is not present, by means of a gas feed line 106. From the pressure swing adsorption stage 104, a hydrogen line 108 leads to the hydroprocessing stage 48, and a fuel gas line 110 is provided. A hydrogen line 109 leads from the hydrogen line 108 to the diesel hydrotreating stage 52.

The optional CO₂ separation stage 96 is optionally connected to the light hydrocarbon recovery stage 36 by means of a line 112, which may instead feed into the DME synthesis stage 98. A compressor 97 is required to increase the pressure to a suitable pressure for DME synthesis. The natural gas feedstock line 14 also branches to the optional CO₂ separation stage 96.

The optional CO₂ separation stage 96 is provided with a CO₂ enriched gas line 114 which is either vented or at least partly returned to the autothermal reforming stage 12. A CO₂ lean gas line 116 leads to the DME synthesis stage 98.

From the DME synthesis stage 98, an aqueous condensate line 118, a tail gas line 120 and a DME product line 122 lead. The tail gas line 120 leads into an optional light hydrocarbon recovery stage 124 from where a light hydrocarbon line 126 joins the DME product line 122 and a fuel gas line 128 is provided, which optionally returns to the pressure swing adsorption stage 104 for hydrogen recovery.

The DME product line 122 is also joined by the light hydrocarbon line 84, after the light hydrocarbon line 84 is joined by a light hydrocarbon line 130 from the optional light hydrocarbon recovery stage 36. The DME product line 122 feeds into a light olefins production stage 132 from where an aqueous condensate line 134 and a light olefins product line 136 lead.

In use, the process 10 is provided with a methane rich gas, such as natural gas, fed along the natural gas feedstock line 14. In the autothermal reforming stage 12, the natural gas is reformed in the presence of oxygen and steam and at a pressure of about 45 bar and a temperature of about 1050° C. Typically, the steam is generated at a pressure of between about 60 bar and about 120 bar, by indirect heat exchange (not shown) with the autothermal reforming stage 12 hot outlet stream before the synthesis gas is removed by means of the line 22. Preferably, a low steam/carbon ratio of 0.4 and optional CO₂ recycle is employed in the autothermal reforming stage 12 to produce a synthesis gas with an H₂/CO ratio in the range of about 1.5 to 2.3, i.e. slightly rich in hydrogen. The synthesis gas thus comprises primarily carbon monoxide, carbon dioxide and hydrogen and includes residual methane and typically small amounts of inert gases, such as nitrogen and argon.

The synthesis gas is fed from the autothermal reforming stage 12 to the Fischer-Tropsch reaction stage 20 by means of the syngas feed line 22. Although not shown in the drawings, the synthesis gas is typically first cooled in an air cooler to a temperature of about 70° C., before being fed into the Fischer-Tropsch reaction stage 20. During this air cooling of the synthesis gas, an aqueous condensate is produced, which is removed from the synthesis gas.

The Fischer-Tropsch reaction stage 20 comprises one or more slurry phase reactors, operating at a pressure between 10 bar and 50 bar, typically about 30 bar and a temperature typically between 220° C. and 260° C. These three-phase slurry phase reactors each include a slurry bed of solid particulate promoted iron Fischer-Tropsch catalyst suspended in liquid hydrocarbon product (mostly wax). The synthesis gas enters the slurry beds at a low level and the hydrogen and carbon monoxide react catalytically as they pass upwardly through each slurry bed, thereby to form liquid hydrocarbon products and gaseous products. The liquid product is withdrawn along the liquid hydrocarbon line 64 and fed to the hydroprocessing stage 48. The gaseous products and unreacted synthesis gas feedstock leave the Fischer-Tropsch reaction stage 20 along the gaseous product line 24. In the air cooler 26 the gaseous products and unreacted feedstock are cooled, producing an aqueous condensate which is removed along the aqueous condensate line 32, from the three-phase separation stage 28 where it is separated. A hydrocarbon condensate is removed by means of the hydrocarbon condensate line 38 from the separation stage 28 and fed to the fractionation stage 40. The three-phase separation stage 28 also produces a tail gas which is withdrawn along the tail gas line 34.

A portion of the tail gas is recycled by means of the tail gas recycle line 86 and the compressor 88 to the Fischer-Tropsch reaction stage 20. A ratio of recycled tail gas to fresh synthesis gas of about 1:1 is maintained. In the Fischer-Tropsch reaction stage 20, overall CO and hydrogen conversion is maintained at about 50%. Per pass conversion is thus about 30%. This is advantageous, as water is formed in the Fischer-Tropsch reaction stage 20 and water deactivates the iron catalyst. Thus, by keeping the per pass conversion low, the partial pressure of the water is kept sufficiently low, i.e. less than 3 bar, to ensure that the catalyst activity remains at a reasonably high level.

A portion of the tail gas from the three-phase separation stage 28 is fed to the DME synthesis stage 98 by means of the line 112, typically being compressed by the compressor 97 to about 100 bar. Before compression and entering the DME synthesis stage 98, this portion of tail gas may however be subjected to a light hydrocarbon recovery operation in the optional light hydrocarbon recovery stage 36, in order to recover light hydrocarbons such as C₂ to C₅ hydrocarbons and traces of C₆ hydrocarbons, which may then be removed along the light hydrocarbon line 130.

The portion of the tail gas fed to the DME synthesis stage typically has a syngas number which is less than 2 because of the presence of excess CO₂. Preferably, for DME synthesis, the syngas number should be about 2 and it is thus desirable that the composition of the tail gas be adjusted prior to the tail gas entering the DME synthesis stage 98. The composition of the tail gas can be adjusted by either removing excess CO₂ or adding a hydrogen-rich gas to the tail gas.

In the process 10 shown in FIG. 1 of the drawings, the composition of the tail gas can be adjusted for DME synthesis purposes by the removal of CO₂. This option is illustrated by the CO₂ separation stage 96 into which the portion of the tail gas intended for DME synthesis is fed by means of the line 112 between the light hydrocarbon recovery stage 36 and the CO₂ separation stage 96. In the CO₂ separation stage 96, the tail gas is contacted with a solvent, such as a Benfield solution, which absorbs some of the CO₂, providing a CO₂ lean gas which can be fed by means of the CO₂ lean gas line 116 to the DME synthesis stage 98.

The CO₂ removed from the tail gas may be recovered by stripping it from the solvent. Typically, this is achieved by using a stripping gas (in this case the natural gas feedstock) and elevating the temperature of the solvent, producing a CO₂ rich gas.

By means of the CO₂ rich gas line 114, the CO₂ rich gas is either vented or at least partly recycled to the autothermal reforming stage 12. This recycle facility allows for better control of the H₂/CO ratio in the synthesis gas fed to the reaction stage 20 thereby favourably to decrease methane selectivity in the Fischer-Tropsch reaction stage 20 and increase the olefin content of the hydrocarbons from the Fischer-Tropsch reaction stage 20.

As mentioned hereinbefore, another option available to adjust the syngas number of the DME synthesis feedstock is to add a hydrogen rich gas to the DME synthesis feedstock. This option can easily be entertained when the steam reforming stage 90 is present and is illustrated by means of the H₂ rich gas line 92 joining the line 112 between the light hydrocarbon recovery stage 36 and the DME synthesis stage 98.

The hydrogen rich gas is obtained from the steam reforming stage 90, which is fed with natural gas along the natural gas feedstock line 14 and with high pressure steam by means of the steam feed line 100. In the steam reforming stage 90, the methane-containing natural gas and the steam are mixed and passed at an elevated temperature and pressure, through externally heated tubes containing a suitable steam reforming catalyst. Catalysts employed are typically nickel on a suitable support, e.g. alumina, magnesia, zirconia, or calcium-aluminate cement. The tubes are heated by a suitable gas, typically the product of combusting a fuel gas. Typically, the temperature is in the range of 700° C. to 950° C., and the pressure is in the range of 15 to 50 bar, particularly 40 bar. The steam is normally present in an excess over that required for the reforming reaction in order to reduce the risk of formation of carbon deposits on the reforming catalyst. The reformed gas will contain hydrogen, carbon monoxide, carbon dioxide, unreacted steam and methane. Although not shown in FIG. 1 of the drawings, the reformed gas is usually cooled to below the dew point of the steam therein to condense the unreacted steam which is then separated, leaving the residual reformed gas which is a hydrogen rich gas.

For some operations, a gas even more rich in hydrogen than the H₂ rich or reformed gas from the steam reforming stage 90 is required. This gas can be obtained by subjecting the H₂ rich or reformed gas to a water-gas shift reaction, as is illustrated by the water-gas shift reaction stage 94. In the water-gas shift reaction stage 94, the H₂ rich gas is mixed with steam fed by means of the steam feed line 102 and passed over a suitable shifting catalyst which promotes the water-gas shift reaction. Some of the carbon monoxide and steam is thus converted to carbon dioxide and hydrogen, thereby further enriching the H₂ rich gas in hydrogen. This further enriched gas is fed by means of the gas feed line 106 into the pressure swing adsorption stage 104 where by means of conventional pressure swing adsorption a hydrogen stream is produced which can be fed by means of the hydrogen line 108 to the hydroprocessing stage 48 and by means of the hydrogen line 109 to the diesel hydrotreating stage 52. A fuel gas produced by the pressure swing adsorption stage 104 is removed by means of the fuel gas line 110. This fuel gas can be used for heating purposes in other areas of the process 10, e.g. the steam reforming stage 90.

In the DME synthesis stage 98, the DME synthesis feedstock is converted to DME by passing the DME synthesis feedstock over a methanol catalyst and a methanol dehydration catalyst, providing a product mixture of DME and methanol. The product mixture is rectified to the required purity and the excess methanol is recycled. The DME synthesis stage 98 is preferably operated at a pressure of at least 25 bar preferably above 90 bar and a temperature as low as economically feasible, to promote methanol formation and methanol dehydration. As mentioned hereinbefore, it is typically necessary to compress the DME synthesis feedstock, by means of the compressor 97, before passing the DME synthesis feedstock to the DME synthesis stage 98. Typically, a copper-containing catalyst is used to produce methanol from the DME synthesis feedstock, whereas the methanol dehydration catalyst is typically a catalyst comprising alumina or alumina silicates as active compounds.

Advantageously, the DME product can be converted to light olefins in the light olefins production stage 132. The DME product is thus fed to the light olefins production stage 132 by means of the DME product line 122 and passed over a DME dehydration catalyst, such as ZSM-5 or SAPO-34. In the process, the DME is dehydrated, producing an aqueous condensate stream which is removed along the aqueous condensate line 134, and a light olefins product which is removed along the light olefins product line 136. This light olefins product typically includes ethylene and propylene and may include aromatics and light paraffins such as methane and propane.

A tail gas from the DME synthesis stage 98 is fed by means of the tail gas line 120 into an optional light hydrocarbon recovery stage 124 to recover at least C₃+ light hydrocarbons, which are combined with the DME product in the DME product line 122, by means of the light hydrocarbon line 126, for conversion to light olefins in the light olefins production stage 132. The light hydrocarbon recovery stage 124 also produces a fuel gas, which is removed along the fuel gas line 128 for use in other locations, or which is optionally recycled for hydrogen recovery to the pressure swing adsorption stage 104 by means of the fuel gas line 128.

The hydrocarbon condensate removed from the three-phase separation stage 28 by means of the hydrocarbon condensate line 38 is subjected to fractionation in a fractionation stage 40 (atmospheric distillation) to produce a heavies stream which is passed to the hydroprocessing stage 48 by means of the heavies lines 46, a diesel fraction which is fed to the diesel hydrotreating stage 52 by means of the diesel fraction line 50 and a light hydrocarbon fraction which is fed to the light olefins production stage 132 by means of the light hydrocarbon line 84. A kerosene fraction, e.g. a C₁₀ to C₁₄ fraction, is removed from the fractionation stage 40 by means of the kerosene fraction line 42 and fed to the alkylation stage 44, where it is treated to remove oxygenated hydrocarbons and then alkylated in the presence of benzene to produce a linear alkyl benzene product. The benzene is fed by means of the benzene feed line 54 into the alkylation stage 44 and a paraffin/oxygenates mixture is removed by means of the paraffin/oxygenates feed line 58, which is fed into the diesel hydrotreating stage 52. Linear alkyl benzene product is removed by means of the linear alkyl benzene product line 62 from the alkylation stage 44.

A naphtha fraction, e.g. a C₅ to C₈ or Cg fraction is removed from the fractionation stage 40 by means of the naphtha fraction line 30 and fed to a comonomer separation stage 56 from which comonomer products, e.g. 1-hexene and 1-octene (C₆ and C₈ alpha olefins), are removed by means of comonomer product lines 60 (only one of which is shown). The remaining naphtha is fed to the light hydrocarbon line 84 by means of the naphtha line 57.

In the diesel hydrotreating stage 52, the diesel fraction from the fractionation stage 40 and the paraffins and oxygenates remaining after the alkylation stage 44 are subjected to hydrotreatment, with the hydrogen being fed by means of the line 109 from the pressure swing adsorption stage 104. A diesel product is thus produced from paraffins, olefins and oxygenates and removed from the diesel hydrotreating stage 52 by means of the diesel line 72.

In the hydroprocessing stage 48, the liquid hydrocarbon wax from the Fischer-Tropsch reaction stage 20 and the heavy hydrocarbons from the fractionation stage 40 are subjected to hydroprocessing. The hydrogen is fed from the pressure swing adsorption stage 104 by means of the hydrogen line 108 into the hydroprocessing stage 48. The hydroprocessing stage 48 produces a naphtha product which is removed by means of the naphtha line 66 and sent to the light olefins production stage 132. A diesel product from the hydroprocessing stage 48 is removed by means of the diesel line 70. Unconverted heavies from the hydroprocessing stage 48 is removed by means of the unconverted heavies line 74 and recycled. The hydroprocessing stage 48 produces a range of valuable lubricating oils and other waxes and products, which are represented by the lubricating oils product line 82.

Referring to FIG. 2 of the drawings, a process in accordance with the invention for producing liquid hydrocarbon fuels, DME and light olefins, is generally indicated by reference numeral 200. The process 200 is similar to the process 10, and unless otherwise indicated, the same reference numerals are used to indicate the same or similar parts or features. The process 200 illustrates the invention for the case where the gaseous feedstock is derived from a solid carbonaceous material, such as coal.

The process 200 includes a gasification stage 202 supplied with a coal feedstock, indicated by reference numeral 204. A raw gas line 206 connects the gasification stage 202 to a CO₂ and sulphur removal stage 208 from where the syngas feed line 22 leads into the Fischer-Tropsch reaction stage 20.

As will be noted in FIG. 2, the light hydrocarbon recovery stage 36 is optionally followed by a slipstream water-gas shift reaction stage 94, which is thus optionally located between the light hydrocarbon recovery stage 36 and the CO₂ separation stage 96. A CO₂ gas line 210 leads from the CO₂ separation stage 96.

The process 200 is operated in similar fashion to the process 10, but with a few alterations. Instead of a methane-containing gas providing the hydrocarbon feedstock, the hydrocarbon feedstock is derived from coal which is fed to the gasification stage 202 and gasified in a plurality of conventional gasifiers, such as Lurgi (trade name) gasifiers. The gasifiers typically produce a gaseous carbonaceous feedstock with an H₂/CO molar ratio of between about 0.7 and about 2.1, more typically between about 1.9 and 2.1 when Lurgi (trade name) gasifiers are being used. The raw gas from the gasification stage 202 is fed by means of the raw gas line 206 to the CO₂ and sulphur removal stage 208 to produce a synthesis gas suitable for Fischer-Tropsch hydrocarbon synthesis. The synthesis gas is then fed by means of the synthesis gas feed line 22 into the Fischer-Tropsch reaction stage 20 from where the process 200 is very similar to the process 10 in respect of the stages 26, 28, 40, 44, 48, 56, 52, 36, 98, 124 and 132. However, the optional slip-stream water-gas shift reaction stage 94 is provided after the light hydrocarbon recovery stage 36 with the purpose of enriching the tail gas being fed to the DME synthesis stage 98 with hydrogen. Thus, in the water-gas shift reaction stage 94, carbon monoxide is reacted with steam to produce carbon dioxide and hydrogen, whereafter some of the carbon dioxide is removed in the CO₂ separation stage 96 to produce a carbon dioxide stream indicated by reference numeral 210 and a CO₂ lean gas, which is in effect hydrogen enriched, and which is combined by means of the CO₂ lean gas line 116 with the tail gas in the line 112 between the light hydrocarbons recovery stage 36 and the DME synthesis stage 98 to provide a DME synthesis feedstock with a syngas number of about 2.

It is an advantage of the process 10, 200, that DME, naphtha and LPG are co-processed to produce light olefins such as ethylene and propylene. The process 10, as illustrated, is also quite flexible as far as the relative production of Fischer-Tropsch hydrocarbons and DME or light olefins is concerned, with the production of one versus the production of the other easily being increased or decreased, depending on where most value can be derived from. The process also allows for conversion of some of the CO₂ produced by synthesis gas production and Fischer-Tropsch reaction to useful products by means of DME synthesis if hydrogen is available. Overall carbon efficiency is thus improved and CO₂ emissions reduced. The processes 10, 200, as illustrated, also allow less severe light hydrocarbon recovery in the light hydrocarbon recovery stage 36, than is the case for a conventional Fischer-Tropsch synthesis process aimed only at producing Fischer-Tropsch derived hydrocarbons, if the light hydrocarbons can be fed through the DME synthesis stage 98 without adverse effects. The process 10, 200, as illustrated, enjoys the benefits of Fe-based Fischer-Tropsch catalysts, whilst simultaneously, as a result of the DME synthesis stage, avoiding the disadvantages of using Fe-based Fischer-Tropsch catalysts.

It is believed that partially converting synthesis gas to hydrocarbons in a first low temperature Fischer-Tropsch reaction stage yields more valuable products than merely converting the synthesis gas to a DME product. It is also believed that converting the tail gas from the first stage low temperature Fischer-Tropsch reaction stage using a DME synthesis stage is more advantageous than employing a second low temperature Fischer-Tropsch reaction stage. This is partly due to the fact that carbon dioxide is reactive in the DME synthesis stage and reacts to form products. A further consideration is the low reactor productivity that is achievable in the second Fischer-Tropsch reactor. This is economically undesirable. On the other hand, the reactor productivity in the second stage DME synthesis is similar to the reactor productivity for stand alone DME synthesis. In addition, in a DME synthesis stage the chemical reaction equilibrium that limits the achievable conversion for a pure methanol synthesis is to some extent alleviated by converting to DME.

### EXAMPLE 1

A process comprising the series combination of two three-phase low temperature Fischer-Tropsch reaction stages was modelled using a computerised simulation to set a base case for comparison with the improvement derived from the present invention.

Both Fischer-Tropsch reactors are operated at a pressure of 30 bar and a temperature of 240° C. Both reactors are loaded with an equal amount of promoted iron Fischer-Tropsch catalyst. The catalyst is therefore a shifting catalyst.

A process with a natural gas feedstock was considered. A typical synthesis gas composition ex an autothermal reformer was used as fresh synthesis gas, i.e. a molar composition of 64.3% hydrogen, 28.6% carbon monoxide, 3.3% carbon dioxide, 2.3% methane and 1.5% inerts.

The fresh synthesis gas is mixed with recycled synthesis gas from the first Fischer-Tropsch reaction stage tail gas and fed to the first Fischer-Tropsch reaction stage. The liquid hydrocarbon product (wax) is removed from the reactor and processed further. The gaseous effluent from the reactor is cooled to 30 to 70° C. to condense hydrocarbon condensate and water. The uncondensed tail gas is split into a recycle stream and a purge stream. The recycle stream is admixed with the fresh synthesis gas. The purge stream is the feed to the second Fischer-Tropsch reaction stage.

The first reaction stage is set to target an H₂ and CO conversion of 50.4% at a per pass conversion of 29.9%. This is achieved at a recycle ratio of 0.8. The water partial pressure in the reactor outlet is 2.7 bar. The overall CO and CO₂ conversion achieved in the first Fischer-Tropsch reaction stage is 47.8%.

The second reaction stage is operated similarly to the first stage. The second reaction stage operates at a per pass H₂ and CO conversion of 31.9%, a recycle ratio of 1.1 and an overall H₂ and CO conversion of 59.8% for the second reaction stage. The water partial pressure in the reactor outlet is 2.4 bar. The overall CO and CO₂ conversion achieved over the second Fischer-Tropsch reaction stage is 53.5%.

The H₂ and CO conversion achieved in the overall process is 79.8%, with a total CO and CO₂ conversion of 75.5%. The actual yield of the process to the maximum theoretical yield is 72%.

The overall process produces a product slate of wax (C₂₄+):C₁₃-C₂₃ condensate:C₇-C₁₂ condensate of 2.95:1.67:1.

### EXAMPLE 2

In a comparative example to illustrate the benefits of the present invention, a process in which a natural gas-based feed is partially converted to hydrocarbons in a three-phase low temperature Fischer-Tropsch reaction stage and the tail gas converted to DME product in a DME synthesis stage, was modelled using a computerised simulation.

An identical fresh synthesis gas composition to Example 1 was used.

The operation, conversion and product yield from the Fischer-Tropsch reaction stage are identical to that of the first Fischer-Tropsch reaction stage in Example 1.

The syngas number of the purge gas from the Fischer-Tropsch reaction stage is adjusted upwardly to 2.03 by partial removal of CO₂. The syngas number adjusted gas is then compressed and fed to the DME synthesis stage.

The simulated DME synthesis stage consists of a cooled methanol reactor followed by an adiabatic combined methanol synthesis and dehydration reactor that contains a bed of dual function catalyst (i.e. combined methanol formation and methanol dehydration) and a bed of methanol dehydration catalyst. The process operates at a pressure of 100 bar.

Recycled synthesis gas is mixed with syngas number adjusted tail gas from the Fischer-Tropsch reaction stage and preheated to 225° C. 15% of the preheated stream is split from the preheated stream (forming a bypass stream) prior to feeding the remaining 85% to the methanol reactor. The outlet temperature from the methanol reactor is controlled to 274° C. The effluent from the methanol reactor is mixed with the bypass stream and fed to the combined methanol synthesis and dehydration reactor. The effluent from the combined methanol synthesis and dehydration reactor is cooled to condense approximately 99% of the water and methanol and 20% of the DME. The uncondensed gas is split into a recycle stream and a purge stream. The recycle stream is admixed with the syngas number adjusted tail gas from the Fischer-Tropsch reaction stage. The purge stream is subjected to an additional cooling step to remove all of the DME.

The DME synthesis stage is operated with a per pass H₂ and CO conversion of 22.2%, a recycle ratio of 3.1, an overall H₂ and CO conversion over the DME synthesis stage of 75.6% and an overall CO and CO₂ conversion over the DME synthesis stage of 79%. A product mass ratio of DME:methanol of 2:1 is achieved.

The overall process has an overall H₂ and CO conversion of 88% and an overall CO and CO₂ conversion of 90.1%.

The mass ratio of products for methanol:DME:wax (C₂₄+):C₁₃-C₂₃ condensate:C₇-C₁₂ condensate is 3.4:7:3.6:1.9:1.

The actual yield of the process to the maximum theoretical yield is 83%.

When the results for Example 2 are compared to the results for Example 1, it is clear that an improved overall CO and CO₂ conversion is achievable for the combination of low temperature Fischer-Tropsch synthesis and DME synthesis when compared to the series combination of low temperature Fischer-Tropsch synthesis stages, since CO₂ is reactive in DME synthesis to form products.

While it is possible to increase the second stage conversion in Example 1 by increasing recycle in order to increase the overall process yields, this will be accompanied by a decline in reactor productivity. The second stage Fischer-Tropsch reactor productivity in Example 1 is already significantly lower than the first stage Fischer-Tropsch reactor productivity. Reactor productivity means the products produced per unit of reactor or catalyst volume. This is economically undesirable. On the other hand, the reactor productivity in the second stage DME synthesis in Example 2 is similar to the reactor productivity for stand alone DME synthesis. Hence the combined process of Example 2 provides a higher yield in comparison to each individual process while achieving similar or higher reactor productivity.

1. A process for co-producing hydrocarbons and dimethyl ether (DME), the process including
feeding a gaseous feedstock comprising hydrogen and carbon monoxide, into a three-phase low temperature catalytic Fischer-Tropsch reaction stage which includes a slurry bed of a solid particulate shifting Fischer-Tropsch catalyst suspended in a carrier liquid;
allowing the hydrogen and carbon monoxide partially to react catalytically in the Fischer-Tropsch reaction stage to form hydrocarbons, at an overall CO and H₂ conversion of between 30% and 60%;
obtaining a tail gas from the Fischer-Tropsch reaction stage which includes unreacted hydrogen and carbon monoxide and also carbon dioxide;
adjusting the composition of at least a portion of the tail gas to provide a DME synthesis feedstock with a syngas number (SN) between 1.8 and 2.2, where
$$SN = \frac{[ {H}_{2} ] - [ {CO}_{2} ]}{[ CO ] + [ {CO}_{2} ]}$$
and where [H₂], [CO] and [CO₂] respectively are the molar proportions of hydrogen, carbon monoxide and carbon dioxide in the DME synthesis feedstock;
feeding the DME synthesis feedstock into a DME synthesis stage; and
converting at least a portion of the DME synthesis feedstock fed to the DME synthesis stage to DME.

2. The process as claimed in claim 1, in which the syngas number is between 1.85 and 2.15.

3. The process as claimed in claim 1, in which adjusting the composition of at least a portion of the tail gas from the Fischer-Tropsch reaction stage includes removing some CO₂ from said portion of the tail gas, thereby adjusting the syngas number upwardly.

4. The process as claimed in claim 3, in which removing some CO₂ from said portion of the tail gas from the Fischer-Tropsch reaction stage includes absorbing the CO₂ in a solvent, and recovering the removed CO₂, by stripping the CO₂ from the solvent.

5. The process as claimed in claim 4, in which the CO₂ is stripped from the solvent with a methane-containing gas and in which the gaseous feedstock to the Fischer-Tropsch reaction stage is derived from the methane-containing gas.

6. The process as claimed in claim 1, in which adjusting the composition of at least a portion of the tail gas from the Fischer-Tropsch reaction stage includes adding an H₂ rich gas to said portion of the tail gas.

7. The process as claimed in claim 6, in which adding an H₂ rich gas to said portion of the tail gas from the Fischer-Tropsch reaction stage includes reforming a portion of the gaseous feedstock to the Fischer-Tropsch reaction stage in a steam reforming stage to produce an H₂ rich reformed gas, and combining at least some of the H₂ rich reformed gas with said portion of the tail gas to provide the DME synthesis feedstock.

8. The process as claimed in claim 6, which includes removing an H, containing tail gas from the DME synthesis stage, recovering an H₂ rich gas from the DME synthesis stage tail gas, and adding said H₂ rich gas to the portion of the tail gas from the Fischer-Tropsch reaction stage to provide the DME synthesis feedstock.

9. The process as claimed in claim 6, in which adding an H₂ rich gas to said portion of the tail gas CO+H₂O⇄CO₂+H₂
from the Fischer-Tropsch reaction stage includes subjecting a synthesis gas to the water gas shift, and removing at least some of the CO₂ to provide the H₂ rich gas, and combining at least some of the H₂ rich gas with said portion of the tail gas to provide the DME synthesis feedstock.

10. The process as claimed in claim 1, in which some of the tail gas from the Fischer-Tropsch reaction stage is recycled to the Fischer-Tropsch reaction stage.

11. The process as claimed in claim 1, which includes treating the hydrocarbons to provide a naphtha fraction and/or a kerosene fraction.

12. The process as claimed in claim 1, in which the Fischer-Tropsch reaction stage is operated at a temperature of less than 280° C. so that the liquid hydrocarbon product from the Fischer-Tropsch reaction stage comprises predominantly wax.

13. The process as claimed in claim 1, which includes withdrawing liquid hydrocarbon products and gases and vapours from the Fischer-Tropsch reaction stage and cooling the gases and vapours to condense liquid hydrocarbons and reaction water present therein and in which a kerosene fraction of the condensed liquid hydrocarbons from the Fischer-Tropsch reaction stage is treated to remove oxygenated hydrocarbons and then alkylated and subjected to a separation stage to produce linear alkyl benzene, and optionally paraffins and oxygenates.

14. The process as claimed in claim 1, in which converting at least a portion of the DME synthesis feedstock fed to the DME synthesis stage to DME includes contacting the DME synthesis feedstock with catalyst or catalysts that enhance methanol synthesis and methanol dehydration reactions, thereby to produce DME.

15. A process for co-producing a liquid fuel and light olefins, the process including
co-producing liquid hydrocarbons and dimethyl ether (DME) from a gaseous feedstock comprising hydrogen and carbon monoxide, the liquid hydrocarbons and the DME being co-produced in accordance with the process as claimed in claim 1;
treating the liquid hydrocarbons to provide a liquid fuel; and
converting at least some of the DME into light olefins.

16. The process as claimed in claim 15, in which treating the liquid hydrocarbons to provide a liquid fuel includes subjecting at least a portion of the liquid hydrocarbons to hydroprocessing, thereby to produce lubricants and a diesel fraction, the liquid fuel produced thus being Fischer-Tropsch derived liquid fuel and including a diesel fraction.

17. The process as claimed in claim 15, in which the liquid hydrocarbons are also treated to produce a naphtha fraction and optionally a kerosene fraction, and in which at least some of the naphtha fraction and optionally some of the kerosene fraction are converted with at least some of the DME into light olefins.
